(19) United States
(12) Patent Application Publication
(10) Pub. No.: US 2007/0123586 A1
(43) Pub. Date: May 31, 2007

(54) Nitroso derivatives of diphenylamine

(76) Inventors: Claude Lardy, Lyon (FR); Phillippe Guedat, Montenois (FR); Lidia Caputo, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/580,413

(22) PCT Filed: Nov. 17, 2004
(86) PCT No.: PCT/EP04/14892

§ 371 Date: May 24, 2006

(30) Foreign Application Priority Data
Nov. 27, 2003 (FR) 03/13953

(51) Int. Cl. A61K 31/277; A61K 31/195; A61K 31/13

(52) U.S. Cl. 514/522; 514/567; 514/611; 558/419; 562/435

(57) ABSTRACT

The invention relates to a compound of the formula I 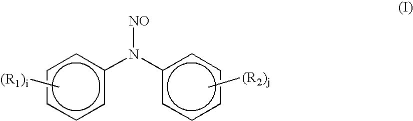 in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an alkylcarbonyl group; a carboxyl group; an alkylcarbonylamino group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group; i and j independently being 1 to 5,
   with the exclusion of the compound for which i and j=1 and R₁=carboxyl and R₂=alkoxycarbonyl or R₁=CF₃ and R₂=carboxyl, and also the pharmaceutically acceptable derivatives, salts, solvates and stereoisomers thereof, including mixtures thereof in all proportions.

The invention relates to nitroso derivatives of diphenylamine, to pharmaceutical compositions comprising them and to the use thereof for the preparation of medicaments that can be used for the treatment of pathologies characterized by an oxidative stress condition and a lack of availability of endothelial nitrogen monoxide (NO.).

Nitrogen monoxide (or nitric oxide NO.) is an important mediator in the physiology of cardiovascular, immune and central and peripheral nervous systems. It acts especially by activation of guanylate cyclase.

Its action is ubiquitous: it is vasodilatory and gives a basal tonus to the entire vascular system. It has anti-clotting activity: its production by normal endothelial cells inhibits the formation of a thrombus. It is anti-proliferative, especially on the smooth muscle cells underlying the endothelial cells. It also inhibits the adhesion of monocytes to the vascular wall and, consequently, their conversion into macrophages. It regulates endothelial permeability.

There is thus, in the physiological state, a state of equilibrium between the production of free-radical species and the availability of NO.

Disequilibrium of this balance, the result of which is an excess of superoxide anions in the face of a lack of NO, leads to the development of many pathologies.

Oxidative stress is generated by many factors, for instance hyperglycaemia, dyslipidaemia (production of oxidized, highly atherogenic “low-density” lipoproteins (LDL)), hypoxia, insulin resistance, atherosclerosis, revascularization techniques (including angioplasties with or without a stent), chronic rejection after transplantation, the majority of inflammatory processes, and smoking. Oxidative stress is characterized at the vascular level by an increase in free radicals, in particular in superoxide anions (O₂.⁻).

These O₂.⁻ radicals are capable of trapping the NO endogenously produced by the endothelial cells to form free-radical species that are even more deleterious, for instance peroxynitrites.

Among the pathologies concerned by a lack of production of endothelial nitrogen monoxide and/or an increase in tissue oxidative stress, mention may be made of (Recent Progress in Hormone Research (1988), 53, 43-60, table V):
atherosclerosis-related ischaemias (lipid peroxidation, development, progress and rupture of atheroma plaques, platelet activation);
restenosis after angioplasty;
stenosis after vascular surgery;
diabetes;
insulin resistance;
retinal, renal and neuronal microvascular complications of diabetes, and also diabetes-related ulcers of the lower limbs;
the cardiovascular risk of diabetes that is only partially explained by the conventional factors;
male erectile dysfunction;
pulmonary arterial hypertension;
cerebral hypoxia;
chronic rejection after organ transplantation;
cold ischaemia during organ transplantation;
extracorporeal circulation;
articular pathologies.

In the context of these pathologies, an ensemble of impairments representing cardiovascular risk factors has been combined under the term “syndrome X” or “metabolic insulin-resistance syndrome” (MIRS) (Reaven G M: Role of insulin resistance in human disease, Diabetes 1988; 37: 1595-607); it includes insulin resistance, hyperinsulinism, glucose intolerance or declared diabetes, arterial hypertension and hypertriglyceridaemia.

Other anomalies are frequently associated with this syndrome: android obesity, microalbuminia, hyperuricaemia, clotting anomalies and fibrinolysis anomalies. Hepatic steatosis of non-alcoholic origin may also be associated therewith.

The administration of active principles capable of reducing the biological activity of oxidative free-radical species (such as superoxide anions and peroxynitrites) and of increasing the content of nitrogen monoxide by a twofold mechanism: non-conversion into peroxynitrites and exogenous supply, is thus particularly desirable in the treatment of these pathologies.

The present invention provides compounds that have both an antioxidant effect and a nitrogen monoxide-donating effect, which are capable of spontaneously generating nitrogen monoxide under physiological conditions and of trapping oxidative free radicals.

The spontaneous NO-donating effect does not induce a tachyphylactic effect, unlike compounds that are substrates of NO synthase, and unlike nitro derivatives or derivatives of oxadiazole or oxatriazole type which mobilize endogenous thiol groups to release NO.

By means of the spontaneous NO-donating effect, pharmacological NO activity may be achieved in pathologies in which the activity of NO synthase is insufficient.

More specifically, the invention relates to the compounds of the formula I: 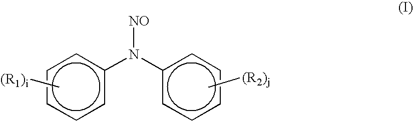
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an alkylcarbonyl group; a carboxyl group; an acylamino group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,
   with the exclusion of the compound for which i and j=1 and R₁=carboxyl and R₂=alkoxycarbonyl or R₁=CF₃ and R₂=carboxyl; and also the pharmaceutically acceptable derivatives, salts, solvates and stereoisomers thereof, including mixtures thereof in all proportions.

The term “halogen atom” means a fluorine, chlorine, bromine or iodine atom, preferably a fluorine or chlorine atom, in particular a fluorine atom.

The term “aliphatic hydrocarbon-based group” means a hydrocarbon-based group with a linear or branched chain containing from 1 to 14 carbon atoms, preferably from 2 to 10 carbon atoms and better still from 2 to 6 carbon atoms, for example from 2 to 4 carbon atoms.

Examples of saturated hydrocarbon-based aliphatic groups are linear or branched (C₁-C₁₀)alkyl radicals, such as methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

These alkyl groups may be substituted, especially with halogen, nitro, cyano, amino, mono- or dialkylamino, carboxyl or acylamino; alkylsulfonyl.

If the hydrocarbon-based aliphatic group is unsaturated, it may comprise one or two unsaturations. The unsaturations are of either ethylenic or acetylenic type. They are preferably ethylenic. The unsaturated chains contain at least two carbon atoms.

Alkenyl and alkynyl groups are examples of unsaturated aliphatic hydrocarbon-based groups.

Examples of unsaturated aliphatic hydrocarbon-based groups are allyl or vinyl.

The expression “optionally interrupted by O and/or S” means that any carbon atom of the hydrocarbon-based chain may be replaced with an oxygen or sulfur atom, this carbon atom not being able to be located at the free end of the hydrocarbon-based chain. The hydrocarbon-based chain, which may be alkyl, may comprise several oxygen and/or sulfur atoms, the hetero atoms preferably being separated from each other by at least one carbon atom and better still by at least two carbon atoms.

An example of an aliphatic hydrocarbon-based chain interrupted by O or S is alkoxy or thioalkoxy.

Examples of halogenated saturated hydrocarbon-based aliphatic groups are haloalkyl groups, such as perhaloalkyl groups of the type —CF₃, —CF₂—CF₃, —CCl₃ or —CCl₂—CCl₃.

Similarly, an example of a halogenated alkoxy group is a perhalo group, such as trifluoromethoxy.

More generally, the substituent R₁ is chosen from halogen atoms and the following groups: cyano; carboxyl; nitro; optionally halogenated (C₁-C₁₄)alkoxy (and preferably methoxy and trifluoromethoxy); optionally halogenated (C₁-C₁₄)thioalkoxy, preferably (C₁-C₁₀)thioalkoxy (and especially thiomethoxy); optionally halogenated and preferably perhalogenated (C₂-C₁₄)alkyl (and especially methyl and trifluoromethyl); (C₁-C₁₄)alkylcarbonyl and especially methylcarbonyl; (C₁-C₁₄)alkoxycarbonyl and especially methoxycarbonyl and ethoxycarbonyl; di(C₁-C₁₀)alkylamino, in particular dimethylamino; and (C₁-C₁₀)alkylsulfonyl, such as methylsulfonyl; and (C₁-C₁₄)alkylcarbonylamino.

The substituent R₂ is advantageously cyano, a hydroxy(C₁-C₁₀)alkyl group, such as CH₂OH; a (C₁-C₁₀)alkylcarbonyl group and especially methylcarbonyl; a carboxyl or (C₁-C₆)alkylcarboxyl group, such as —CH₂COOH, an alkoxycarbonyl group, in particular —COOCH₃ or —COOC₂H₅; and an acylamino or (C₁-C₆)alkylacylamino group.

The two phenyl groups in the compounds of the formula (I) may be substituted one or more times with one or more of the substituents listed above, which may be identical or different, preferably one to three times, for example one or two times.

Advantageously, the compounds of the formula (I) contain only one substituent R₁ and/or only one substituent R₂, respectively, on each of the two phenyl rings. A preferred subgroup of compounds of the formula (I) thus consists of compounds for which i=1 and/or j=1.

The substituents R₁ and R₂ may be located on any one of the ortho, meta or para positions of the phenyl ring.

In addition, the invention relates to the optically active forms (stereoisomers), enantiomers, racemates, diastereoisomers, hydrates and solvates of these compounds. The term “solvate” denotes the adducts of the compounds with inert solvent molecules, which are formed on account of their mutual force of attraction. The solvates are, for example, the monohydrates, dihydrates or alcoholates. The term “pharmaceutically acceptable derivatives” is supposed to denote, for example, the salts of the compounds according to the invention and the compounds known as “prodrugs”.

The term “prodrugs” is defined as denoting, for example, the compounds according to formula (I) that have been modified, for example with alkyl or acyl groups, sugars or oligopeptides, and that are rapidly cleaved in the body to release the active compounds according to the invention.

They also include biodegradable polymer derivatives of the compounds according to the invention, as described, for example, in Int. J. Pharm. 115, 61-67 (1995).

The invention also relates to mixtures of the compounds of the formula (I) according to the invention, for example mixtures of two diastereoisomers, for example in a ratio 1:1, 1:2, 1:3, 1:4, 1:5, 1:10, 1:100 or 1:1000. They are also mixtures of particularly preferred stereoisomeric compounds.

The invention is directed not only towards the compounds of the formula I, but also towards the salts thereof.

If the compound of the formula I comprises an acidic function, for example a carboxylic function, this compound may form a salt with a mineral or organic base.

Examples of salts with organic or mineral bases that may be mentioned include the salts formed with metals and especially alkali metals, alkaline-earth metals and transition metals (such as sodium, potassium, calcium, magnesium or aluminium), or with bases, for instance ammonia or secondary or tertiary amines (such as diethylamine, triethylamine, piperidine, piperazine or morpholine), or with basic amino acids, or with osamines (such as meglumine) or with amino alcohols (such as 3-aminobutanol and 2-aminoethanol).

If the compound of the formula I comprises a basic function, for example a nitrogen atom, this compound may form a salt with an organic or mineral acid.

The salts with organic or mineral acids are, for example, the hydrochloride, hydrobromide, sulfate, hydrogen sulfate, dihydrogen phosphate, nitrate, trifluoroacetate, citrate, maleate, fumarate, 2-naphthalenesulfonate and para-toluenesulfonate.

The invention also covers the salts allowing a suitable separation or crystallization of the compounds of the formula I, such as picric acid, oxalic acid or an optically active acid, for example tartaric acid, dibenzoyltartaric acid, mandelic acid or camphorsulfonic acid.

Formula I includes all the types of geometrical isomers and stereoisomers of the compounds of the formula I.

The compounds of the invention can be prepared simply by reacting a compound of the formula (II): 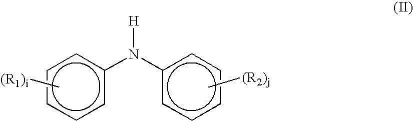
in which R₁, R₂, i and j are as defined for formula (I) above, with a nitrosating agent.

Examples of nitrosating agents that are particularly advantageous include an alkali metal nitrite (and especially sodium or potassium nitrite) or a C₁-C₄ alkyl nitrite.

A preferred alkali metal nitrite that may be mentioned is sodium nitrite.

A preferred alkyl nitrite that may be mentioned is ethyl nitrite.

Nevertheless, a person skilled in the art can use any nitrosating agent known in the art, such as AgONO, BF₄NO, HOSO₃NO, nBuONO or tBuONO.

The amount of nitrosating agent required depends on the nature of the nitrosating agent used and on the reactivity of the substrate of the formula II. It is at least stoichiometric. In general, the molar ratio of the nitrosating agent to the substrate of the formula II ranges between 1 and 30 equivalents and preferably between 1 and 20 equivalents.

If the nitrosating agent is an alkali metal nitrite, a person skilled in the art may readily adapt the reaction conditions so as to use only 1 to 10, preferably from 1 to 5 and better still from 1 to 3 equivalents of nitrite relative to the substrate of the formula II.

If the nitrosating agent is an alkyl nitrite, it is preferable to perform the process in the presence of 10 to 25 molar equivalents of nitrite, and preferably from 15 to 20 molar equivalents, relative to the amount of substrate of the formula II.

The choice of solvent and the temperature conditions depend especially on the type of nitrosating agent selected for the reaction.

If the nitrosating agent is AgONO, nBuONO or tBuONO, the solvent is advantageously chosen from a cyclic or non-cyclic ether (such as diethyl ether, diisopropyl ether, tetrahydrofuran, dioxane, dimethoxyethane or diethylene glycol dimethyl ether), an aliphatic or aromatic halohydrocarbon (such as chloroform, carbon tetrachloride, dichloroethane, chlorobenzene or dichlorobenzene). Preferably, the solvent is tetrahydrofuran, diethyl ether or chloroform.

The reaction temperature will generally be maintained between 15 and 70° C. and better still between 17 and 60° C., in the case of AgONO, nBuONO and tBuONO.

More particularly, in the case of AgONO and nBuONO, the process will be performed in tetrahydrofuran or diethyl ether at a temperature of between 15 and 30° C., for example between 18 and 25° C.

In the case of tBuONO, the process will preferably be performed in chloroform at a temperature of between 40 and 65° C., for example between 50 and 60° C.

If the nitrosating agent is AgONO, it is desirable to add thionyl chloride to the reaction medium.

If the nitrosating agent is HOSO₃NO, the reaction is preferably carried out in an alkali metal salt of a lower (C₁-C₅)carboxylic acid, such as sodium acetate, at a reaction temperature of between −10° C. and 30° C. and better still between −5° C. and 25° C.

If the nitrosating agent is BF₄NO, a suitable solvent is a nitrile, such as acetonitrile or isobutyronitrile. It is desirable to add pyridine or N-dimethylaminopyridine to the reaction medium, the reaction temperature being maintained between −30° C. and 10° C. and preferably between −25° C. and 5° C.

If the nitrosating agent is an alkali metal nitrite, the nitrosation reaction is preferably carried out in a strongly polar protic medium. Advantageously, the reaction medium contains water and a Brönsted or Lewis acid.

Suitable acids are a hydrohalic acid (such as HCl), sulfuric acid, Al₂(SO₄)₃ or acetic acid, and mixtures thereof.

According to a particular embodiment of the invention, an aliphatic alcohol of (C₁-C₄)alkanol type (such as methanol or butanol) may be added.

Thus, a suitable reaction medium that may be selected is one of the following systems:
a mixture of methanol, water, hydrochloric acid and sulfuric acid;
a mixture of water and sulfuric acid;
a mixture of water and acetic acid;
a mixture of water, butanol and hydrochloric acid;
a mixture of water and Al₂(SO₄)₃; or
a mixture of water and hydrochloric acid.

Advantageously, the reaction of the alkali metal nitrite with the substrate of the formula II is carried out in a mixture of acetic acid and water, the ratio of acetic acid to water ranging between 80:20 and 20:80 and preferably between 60:40 and 40:60, for example a 50:50 mixture. According to one preferred embodiment, the alkali metal nitrite, pre-dissolved in water, is added dropwise to a solution of the substrate of the formula II in acetic acid.

The reaction of the alkali metal nitrite with the substrate of the formula II is carried out at a temperature that depends on the reactivity of the species present; this temperature generally ranges between −10° C. and 50° C. and preferably between −5° C. and 25° C.

If the nitrosation reaction is carried out in a mixture of acetic acid and water, a temperature of between 15° C. and 25° C. is particularly suitable.

The reaction of the alkyl nitrite with the substrate of the formula II is preferably carried out in the presence of a C₁-C₄ alkanol in a polar aprotic solvent.

Suitable alkanols that may be mentioned include methanol, ethanol, isopropanol and tert-butanol, ethanol being particularly preferred.

Polar solvents that are preferred are halohydrocarbons, such as methylene chloride, chloroform, carbon tetrachloride, dichloroethane, chlorobenzene or dichlorobenzene; ethers, such as diethyl ether, diisopropyl ether, tetrahydrofuran, dioxane, dimethoxyethane or diethylene glycol dimethyl ether; nitriles, such as acetonitrile or isobutyronitrile; amides, such as formamide, dimethylformamide, dimethylacetamide, N-methyl-2-pyrrolidinone or hexamethylphosphoramide; and mixtures of these solvents in any proportions.

Advantageously, the nitrosation reaction (if an alkyl nitrite is used as nitrosating agent) is carried out in a mixture based on an aliphatic halohydrocarbon and a nitrile, and for example in a 90:10 to 50:50 and preferably a 90:10 to 70:30 mixture of chloroform and acetonitrile, in the presence of ethanol.

The amount of alkanol that needs to be incorporated into the reaction medium is not critical according to the invention. It generally represents 5% to 50% by weight of the reaction medium, and preferably from 5% to 25% by weight.

If the nitrosating agent is an alkyl nitrite, the reaction temperature is generally maintained between −20° C. and 20° C. and preferably between −10° C. and 10° C., for example between 0° C. and 5° C.

According to one preferred embodiment of the invention, a solution of the alkyl nitrite in the alkanol is added dropwise to the substrate of the formula II pre-dissolved in the selected polar solvent.

As a variant, the reaction is carried out in a strongly polar medium consisting of a mixture of a C₁-C₄ aliphatic carboxylic acid ((C₁-C₄)alkyl-COOH), the corresponding acid anhydride and the corresponding alkali metal carboxylate salt, in the presence of P₂O₅. By way of example, a reaction medium consisting of acetic acid, acetic anhydride, potassium acetate and P₂O₅ may be selected. In this case, the reaction temperature is advantageously maintained between 10° C. and 100° C. and preferably between 15° C. and 85° C.

The compounds of the formula II can be prepared by carrying out one of the following processes.

Preparation of the Compounds of the Formula II—Route A—

One method for the preparation of compounds of the formula II consists in reacting a compound of the formula (III) with a compound of the formula (IV)

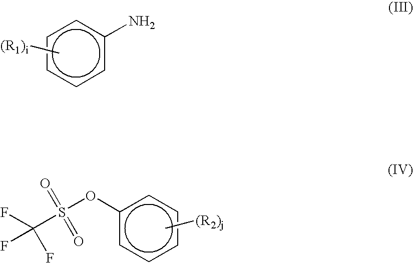

in which R₁, R₂, i and j have the meanings given above.
Advantageously, it is desirable to introduce a palladium-based catalyst into the reaction medium.

Such a catalyst can be obtained by introducing into the reaction medium the system Pd(OAc)₂+BINAP, in which BINAP is the diphosphine of the formula:

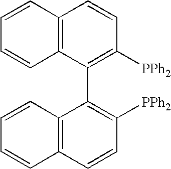

Such a catalyst can also be obtained by introducing into the reaction medium the system (dba)₃Pd₂ (tris(dibenzylideneacetone)dipalladium(0))+BINAP.

Another catalytic system may be composed of Pd(dba)₂ and tri-tert-butylphosphine.

By way of illustration, each of the catalytic substances is introduced into the reaction medium in a proportion of less than 10% by weight. In a particularly advantageous manner, the molar ratio of the BINAP to the (dba)₃Pd₂ or Pd(OAc)₂ ranges between 1 and 3 and preferably between 1.2 and 2.

The molar ratio between the Pd(dba)₂ and tri-tert-butylphosphine is advantageously between 1 and 3 and preferably between 1.2 and 2.

This reaction is preferably performed in the presence of an organic or mineral base. Examples of bases are hydroxides (such as alkali metal hydroxides or ammonium hydroxides), carbonates (such as alkali metal carbonates or ammonium carbonates), alkali metal alkoxides, organic hydrides, alkali metal amides, ammonia and amines, such as triethylamine, tributylamine, pyridine or N-methylmorpholine, among which caesium carbonate or an alkali metal alkoxide is preferred.

This reaction is preferably performed in a nonpolar aprotic solvent, such as toluene or xylene.

The reaction temperature is set as a function of the reactivity of the species present and of the nature of the solvent used. Usually, the temperature ranges between −10° C. and 10° C. Usually, if the base used is an alkali metal or alkaline-earth metal carbonate, the process is performed at the reflux temperature of the solvent. In a particularly advantageous manner, the reaction is performed at a temperature of between 20 and 100° C.

Usually, the molar ratio of compound III to compound IV ranges between 0.8 and 2 and preferably between 0.9 and 1.5, for example between 1.0 and 1.3, a slight excess of compound III possibly being desirable.

The amount of base to be introduced into the reaction medium is generally an excess relative to the molar amount of the compound of the formula III. Preferably, the molar ratio of the base used to compound III ranges between 1 and 2 equivalents, for example between 1.3 and 1.5 equivalents.

One variant comprises the reaction of a compound of the formula (III) with a compound of the formula (IX) 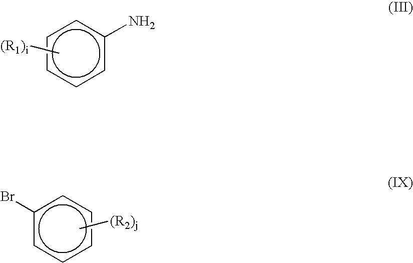
in which R₁, R₂, i and j have the meanings given above.

The reaction conditions are similar to those described above.

Preparation of the Compounds of the Formula II—Route B—

Another process for the preparation of compounds of the formula (II) comprises the reaction of a compound of the formula (V) with a compound of the formula (VI): 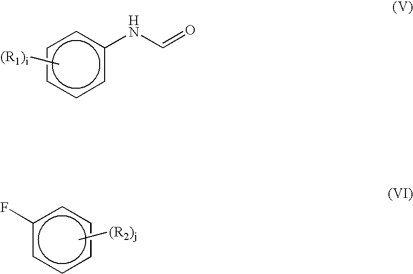
in which R₁, R₂, i and j have the meanings given above.

During this reaction, the fluoro compound VI reacts with compound V, the formyl group of which provides disubstitution. The formyl group is then removed by hydrolysis in basic medium. The base may be an alkali metal hydroxide or hydride or alternatively a base, such as lithium diisopropylamide (LDA), and in particular sodium hydride.

The reaction is advantageously performed by using an amount of base close to the stoichiometric amount. It is thus preferred to have a molar ratio of from 1 to 1.1.

This reaction is preferably performed in a polar aprotic solvent, such as a halogenated hydrocarbon (for example methylene chloride, chloroform, carbon tetrachloride, dichloroethane, chlorobenzene or dichlorobenzene); an ether, such as diethyl ether, diisopropyl ether, tetrahydrofuran, dioxane, dimethoxyethane or diethylene glycol dimethyl ether; a nitrile, such as an acetonitrile or isobutyronitrile; an amide, such as formamide, dimethylformamide, dimethylacetamide, N-methyl-2-pyrrolidinone or hexamethylphosphorylamide; or a ketone, such as acetone or 2-butanone. The solvent is preferably an amide, such as dimethylformamide.

The reaction temperature is set as a function of the reactivity of the species present and of the nature of the solvent used. The temperature usually ranges between −10° C. and 150° C. The process is usually performed at the reflux temperature of the solvent. In a particularly advantageous manner, the reaction is performed in an aprotic solvent, such as dimethylformamide at a temperature of between 120 and 140° C.

Usually, the molar ratio of compound VI to compound V ranges between 0.8 and 2 and preferably between 1 and 1.5, for example between 1.1 and 1.3, a slight excess of compound VI being desirable.

The amide thus obtained is then hydrolysed in a manner that is known per se, to give the compound of the formula II. The hydrolysis is advantageously performed in the presence of a base, such as NaOH. The hydrolysis usually proceeds satisfactorily at room temperature.

Preparation of the compounds of the formula II—Route C—

Another process for the preparation of a compound of the formula (II) comprises the reaction of a compound of the formula (VII) with a compound of the formula (VIII) 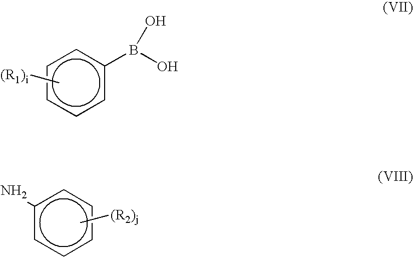
in which R₁, R₂, i and j have the meanings given above.

This reaction is preferably performed in the presence of an organic base. Examples of bases are especially alkali metal alkoxides, organic hydrides and amines, such as triethylamine, tributylamine, pyridine or N-methylmorpholine, triethylamine being particularly preferred.

The reaction takes place in the presence of copper acetate.

This reaction is preferably performed in a polar aprotic solvent, such as a halogenated hydrocarbon (for example methylene chloride, chloroform, carbon tetrachloride, dichloroethane, chlorobenzene or dichlorobenzene); an ether, such as diethyl ether, diisopropyl ether, tetrahydrofuran, dioxane, dimethoxyethane or diethylene glycol dimethyl ether; a nitrile, such as an acetonitrile or isobutyronitrile; an amide, such as formamide, dimethylformamide, dimethylacetamide, N-methyl-2-pyrrolidinone or hexamethylphosphorylamide; or a ketone, such as acetone or 2-butanone. The solvent is preferably methylene chloride.

The reaction temperature is set as a function of the reactivity of the species present and the nature of the solvent used. Usually, the reaction temperature ranges between −10° C. and 100° C. In a particularly advantageous manner, the reaction is performed at room temperature.

Usually, the molar ratio of compound VII to compound VIII ranges between 1 and 6 and preferably between 1.5 and 5, for example between 2 and 4.

The amount of base to be introduced into the reaction medium is generally equivalent to the molar amount of the compound of the formula VIII.

Preparation of the Compounds of the Formula II—Route D—

Yet another process for the preparation of a compound of the formula (II) comprises the reaction of a compound of the formula (III) with a compound of the formula (X) 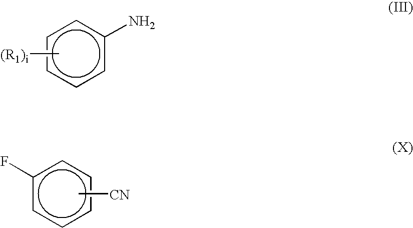
in which R₁ and j have the meanings given above.

The molar ratio of compound III to compound X is generally between 0.8 and 1.2 and preferably about 1.

The coupling is performed in the presence of an organic base chosen from those mentioned in the preceding processes. The amount of base introduced is generally an excess relative to compound III, i.e. between 1 and 2 eq. The solvent used is preferably DMSO.

The reaction temperature depends on the reactivity of the reagents and of the catalytic system used. However, it is generally possible to perform the reaction at room temperature.

The subsequent hydrolysis, under standard conditions, of the nitrile group present on the phenyl ring of the compound obtained then leads to the compounds of the formula II for which R₂ is carboxyl.

According to another of its aspects, the invention relates to a pharmaceutical composition comprising a compound of the formula (I) 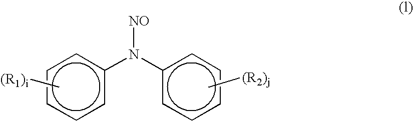
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an acylamino group, an alkylcarbonyl group; a carboxyl group; an unsubstituted amide group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,
   with the exclusion of the compound for which i and j=1 and R₁=carboxyl and R₂=alkoxycarbonyl or R₁=CF₃ and R₂=carboxyl;
   and also the pharmaceutically acceptable derivatives, salts, solvates and stereoisomers thereof, including mixtures thereof in all proportions. The preferred meanings of R¹, R², i and j are those described above.

These compositions can be administered orally in the form of tablets, gel capsules or granules with immediate release or controlled release, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a disintegrant, a lubricant, a colorant or a flavour corrector, and by shaping the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatine, shellac, hydroxypropylcellulose, hydroxypropylmethylcellulose, calcium citrate, dextrin and pectin. Examples of lubricants include magnesium stearate, talc, polyethylene glycol, silica and hardened plant oils. The colorant can be any colorant permitted for use in medicaments. Examples of flavour correctors include cocoa powder, mint in herb form, aromatic powder, mint in oil form, borneol and cinnamon powder. Needless to say, the tablet or granulate can be suitably coated with sugar, gelatine or the like.

An injectable form comprising the compound of the present invention as active principle is prepared, where appropriate, by mixing the said compound with a pH regulator, a buffer agent, a suspending agent, solubilizing agent, a stabilizer, a tonicity agent and/or a preserving agent, and by converting the mixture into a form for intravenous, subcutaneous or intramuscular injection, according to a conventional process. Where appropriate, the injectable form obtained can be freeze-dried by a conventional process.

Examples of suspending agents include methylcellulose, polysorbate 80, hydroxyethylcellulose, acacia, powdered gum tragacanth, sodium carboxymethylcellulose and polyethoxylated sorbitan monolaurate.

Examples of solubilizing agents include castor oil solidified with polyoxyethylene, polysorbate 80, nicotinamide, polyethoxylated sorbitan monolaurate and the ethyl ester of castor oil fatty acid.

In addition, the stabilizer encompasses sodium sulfite, sodium metasulfite and ether, while the preserving agent encompasses methyl p-hydroxybenzoate, ethyl p-hydroxybenzoate, sorbic acid, phenol, cresol and chlorocresol.

According to yet another of its aspects, the invention relates to the use of a compound of the formula (I) 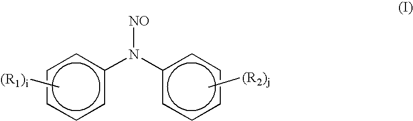
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an acylamino group, an alkylcarbonyl group; a carboxyl group; an unsubstituted amide group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,

and also the pharmaceutically acceptable derivatives, salts, solvates and stereoisomers thereof, including mixtures thereof in all proportions, for the preparation of a medicament that is useful for the treatment of pathologies characterized by an oxidative stress condition and a lack of availability of endothelial nitrogen monoxide.

The nitrogen monoxide-donating effect of the compounds of the invention of the formula I can be simply demonstrated by performing the operating protocol below.

A solution of a compound of the invention of the formula I spontaneously releases nitric oxide. The nitrite ions resulting therefrom are titrated by colorimetry by means of a specific reagent (Griess). To take account of any release of nitrate ions in addition to the nitrite ions, bacterial nitrate reductase is added to the reaction medium, which makes it possible to reduce the nitrate ions formed.

The following tests were performed so as to demonstrate this activity.

The reactions and measurements are performed in transparent 96-well plates. The test products are dissolved extemporaneously at a concentration of 3 mM in dimethyl sulfoxide. 95 μl of a reagent comprising nitrate reductase (0.18 U/ml in 0.100 mM pH 7.5, PBS buffer, β-NADPH 210 μM, FAD 5 μM) and 5 μl of the solution of the test product (final concentration of 150 μM) are then introduced into each well. After stirring, the mixture is incubated for four hours at 37° C. The reaction is then quenched by addition of 100 μl of the Griess reagent (Sigma G4410). This reagent is left to act for five minutes at room temperature, and the optical density is then read at 540 nm. This value is proportional to the concentration of nitrites+nitrates in the medium. A calibration range is made for each plate using NaNO₂.

The results are expressed in μmol/l (μM) of nitrites+nitrates released in Table I for some of the compounds of the formula I illustrated below.

The compounds of the invention of the formula I reduce the biological activity of oxidative free-radical species.

The protocol outlined below was used in order to evaluate this activity.

Human LDLs placed in aqueous solution in the presence of cupric ions, become spontaneously oxidized on their protein component, apolipoprotein-B. This oxidation makes the particle fluorescent, which is exploited to measure a pharmacological effect.

The reactions and measurements are performed in black 96-well plates. 10 μl of a solution of the test product dissolved in dimethyl sulfoxide are first mixed with 170 μl of a solution of human LDL at a concentration of 120 μg/ml and 20 μl of 100 μM CuCl₂. After stirring, the mixture is incubated for 2 hours at 37° C., and a first fluorescence reading is taken (excitation at 360 nm, reading at 460 nm). The mixture is then incubated for a further 22 hours, to take a second reading under the same conditions. The difference is proportionately smaller the greater the antioxidant power of the test product. Probucol is used as reference product at a concentration of 10 μM.

The concentrations that inhibit 50% (IC₅₀) of the oxidation are prepared from three concentrations of the test product. They are given in Table II below for some of the compounds of the formula I given as examples below.

TABLE I
Compounds of the type R₁—Ph—N(NO)—Ph—R₂

| | Nitrites—Nitrates |
|---|---|
| Examples | (μM) |
| 2 | 87 |
| 5 | 108 |
| 6 | 43 |
| 8 | 88 |
| 10 | 76 |
| 12 | 38 |
| 13 | 86 |
| 14 | 69 |
| 15 | 88 |
| 17 | 58 |
| 18 | 81 |
| 19 | 79 |
| 30 | 58 |
| 42 | 79 |

TABLE II
Compounds of the type R₁—Ph—N(NO)—Ph—R₂

| | IC₅₀ antioxidant effect |
|---|---|
| Examples | (μM) |
| 2 | 17.0 |
| 8 | 18.4 |
| 10 | 6.2 |
| 13 | 4.6 |
| 17 | 11.6 |

These compounds of the invention of the formula I also show hypotriglyceridaemiant activity. This activity was especially observed by the inventors on a model of animal pathology.

The compounds were tested on fatty Zucker rats (Zucker L. M. et al., 1961, Fatty a new mutation in the rat, J. Hered., 52: 275-278). This animal is hyperphagic, obese and hyperinsulinaemic. It develops resistance to insulin, it is hyperlipidaemic, and has a large hypertriglyceridaemia.

9-week-old male Zucker rats were treated for eight days with the compounds of Example 2 and Example 5 at a dose of 200 mg/kg/day p.o. After fasting for four hours, a blood sample is taken to recover the plasma. It is found that the compound of Example 2 induces a large reduction in triglycerides, of 58% (p<0.01), and the insulinaemia is down by 47% (p<0.05). The compound of Example 5 has a 21% hypertriglyceridaemiant effect (p<0.05) and reduces insulin by 45% (p<0.05).

The compounds of the formula I of the invention moreover had the effect of reducing the blood contents of free fatty acids and of increasing the blood contents of HDL cholesterol.

The treatment has an effect on the insulinaemia, which is lowered and allows modification of the resistance to insulin.

These properties of the compounds of the invention are useful in the prevention and treatment of diabetes, especially on account of the improvement in the sensitivity to insulin.

More particularly, these compounds can be used for the preparation of a medicament that is useful for the treatment of and preventing diabetes and/or metabolic insulin-resistance syndrome. Moreover, they can be used for the preparation of a hypotriglyceridaemiant medicament.

The present invention is illustrated below in the light of the examples that follow.

The frequency of the NMR machine used to record the proton spectra of the examples proposed below is 300 MHz. The sign s denotes a singlet; d a doublet; t a triplet; q a quartet and m a multiplet. m.p. denotes the melting point.

The LC-MS spectra are obtained on a simple quadrupole machine equipped with an electrospray probe.

### EXAMPLES

### Example 1

### 4-[(4-Methoxyphenyl)(nitroso)amino]benzoic acid

### a) methyl 4-[(4-methoxyphenyl)amino]benzoate

0.303 g (1.35 mmol) of palladium diacetate, 1.04 g (1.69 mmol) of racemic BINAP and then 10.25 g (31.47 mmol) of caesium carbonate are added at room temperature to a mixture of 6.39 g (22.5 mmol) of methyl 4-{[(trifluoromethyl)sulfonyl]oxy}benzoate prepared according to Mowery M. E. and DeShong P. (J. Org. Chem (1999) 64, 3266-3270), 3.32 g (27 mmol) of 4-methoxyaniline and 45 ml of toluene, under nitrogen. The reaction medium is heated at 80° C. for 6 hours. After cooling, the reaction medium is poured into 4 l of water and extracted with ethyl ether. The organic phase is washed with water, dried over Na₂SO₄ and then concentrated to give a brown oil, which, after purification by chromatography on silica gel in CH₂Cl₂, gives 5.38 g of a beige-coloured solid.

Yield: 93.1%

m.p.=88-90° C.

IR (KBr): ν (NH) 3384 cm⁻¹; (CO) 1690 cm⁻

NMR: (CDCl₃): 3.85 (3H, s); 3.90 (3H, s); 6.9-7.1 (4H, m); 7.25 (2H, m); 7.85 (2H, m); 8.6 (1H, s)

### b) 4-[(4-methoxyphenyl)amino]benzoic acid

A mixture of 73.8 g (286 mmol) of the compound prepared in Example 1a, 590 ml of ethanol, 32.1 g (572 mmol) of KOH and 290 ml of water is refluxed for 2 hours.

The reaction medium is then concentrated, taken up in 1600 ml of water, washed with 3×250 ml of ethyl ether and filtered, and is then acidified with acetic acid. The precipitate formed is rinsed with water (3×250 ml) and dried under vacuum, to give 66.6 g of a pink-white solid. After recrystallization from an ethyl acetate/heptane mixture, 55.3 g of a pink-white solid are obtained.

Yield: 79.5%

m.p.=162-164° C.

IR (KBr): ν (NH) 3402 cm⁻¹; (CO) 1673 cm⁻¹

NMR: (DMSO-d6): 3.75 (3H, s); 6.8-7.0 (4H, m); 7.1 (2H, m); 7.7 (2H, m); 8.4 (1H, s, exchangeable with CF₃COOD); 12.2 (1H, broad s, exchangeable with CF₃COOD).

LC-MS:
(ES+)=244.2 (M+H)
(ES−)=242.1 (M−H)

### c) 4-[(4-methoxyphenyl)(nitroso)amino]benzoic acid

A solution of 29.3 g (424 mmol) of sodium nitrite in 210 ml of water is added over 45 minutes to a solution of 51.7 g (212 mmol) of the compound prepared in Example 1b in 2100 ml of acetic acid. A beige-coloured precipitate begins to form. The reaction medium is stirred for 3 hours at room temperature, and then poured into 8 l of cold water. The precipitate is filtered off, rinsed with 3×500 ml of water and dried under vacuum to give 55.28 g of a beige-coloured solid.

Yield: 95.2%

m.p.=169-171° C.

IR (KBr): ν (CO) 1683 cm⁻¹

Elemental analysis: C₁₄H₁₂N₂O₄ (M=272.25)

| | C % | H % | N % |
|---|---|---|---|
| Calculated | 61.76 | 4.44 | 10.29 |
| Found | 61.83 | 4.51 | 10.26 |

NMR: (DMSO-d6): 4.05 (3H, 2s); 7.3 (4H, 2s); 7.4-7.65 (2H, m); 8.2 (2H, m); 13.3 (1H, broad s, exchangeable with CF₃COOD)

LC-MS:
(ES+)=273.2 (M+H); 243.2 (M−NO+H)
(ES−)=271.2 (M−H); 241.2 (M−NO−H)

### Example 2

### 4-[(4-Methoxyphenyl)(nitroso)amino]benzoic acid

### a) 4-[(4-methoxyphenyl)amino]benzoic acid

A solution of 3 g (19.8 mmol) of 4-methoxyphenylformamide, prepared from 4-methoxyaniline according to Ugi I. and Meyr R. (Org. Syntheses, Coll. Vol. 5, 1060-1063), in 7 ml of dimethylformamide (DMF) is added dropwise slowly, at between 10 and 20° C., to a suspension of 0.87 g (21.8 mmol) of NaH at 60% in liquid petroleum jelly, in 3 ml of DMF. After stirring for 30 minutes at room temperature, a solution of 3.5 g (20.7 mmol) of ethyl 4-fluorobenzoate in 5 ml of DMF is added dropwise. The reaction medium is heated at 130° C. for 22 hours. After cooling, 3 ml of 10N HCl solution are added and the reaction medium is concentrated to dryness under vacuum. 40 ml of ethanol, 10 ml of water, 10 ml of THF and 10.6 ml of aqueous 30% NaOH solution are added to the residue obtained. After stirring for 16 hours at room temperature, the reaction medium is concentrated under vacuum. The residue is taken up in 30 ml of water, washed with CH₂Cl₂ (3×30 ml) and acidified to pH 7 with 10N HCl solution. The precipitate formed is filtered off, washed with water and dried under vacuum to give 1.64 g of a beige-coloured solid, which is identical to the product obtained in Example 1 b.

Yield: 34%

### b) 4-[(4-methoxyphenyl)(nitroso)amino]benzoic acid

Obtained by working as in Example 1c.

### Example 3

### 4-[(4-Methoxyphenyl)(nitroso)amino]benzoic acid

### a) ethyl 4-[(4-methoxyphenylamino]benzoate

0.597 g (3.3 mmol) of copper (II) acetate is added to a solution of 0.545 g (3.3 mmol) of ethyl 4-aminobenzoate in 20 ml of CH₂Cl₂, followed by addition of 1 g (6.6 mmol) of 4-methoxyphenylboronic acid and 0.92 ml (6.6 mmol) of triethylamine. After stirring for 18 hours at room temperature, a further 1.19 g (6.6 mmol) of copper (II) acetate, 1 g (6.6 mmol) of 4-methoxyphenylboronic acid and 0.92 ml (6.6 mmol) of triethylamine are added, and stirring is then continued at room temperature for 24 hours. The reaction medium is then poured into water and extracted with CH₂Cl₂. The organic phase is washed with water, dried over Na₂SO₄ and then concentrated and purified by flash chromatography on silica with a (6:1) heptane/ethyl acetate mixture to give 0.543 g of a beige-coloured solid.

Yield: 60.7%

IR (KBr): ν (NH): 3344 cm⁻¹; (CO): 1697 cm⁻¹

NMR: (DMSO-d6): 1.15 (3H, t, J=7.1 Hz); 3.6 (3H, s); 4.1 (2H, q, J=7.1 Hz); 6.8 (4H, m); 7.0 (2H, m); 7.6 (2H, m); 8.4 (1H, s, exchangeable with D₂O)

### b) 4-[(4-methoxyphenyl)amino]benzoic acid

Obtained by working as in Example 1b, starting with the compound obtained in Example 3a.

### c) 4-[(4-methoxyphenyl)(nitroso)amino]benzoic acid

Obtained by working as in Example 1c.

### Example 4

### 4-[(4-Methoxyphenyl)(nitroso)amino]benzoic acid

### a) ethyl 4-[(4-methoxyphenyl)amino]benzoate

1.76 ml (0.176 mmol) of a 0.1 M solution of tri-tert-butylphosphine in toluene are added to a mixture composed of 2.52 g (11 mmol) of ethyl 4-bromobenzoate, 1.354 g (11 mmol) of 4-methoxyaniline and 0.128 g (0.22 mmol) of bis(dibenzylideneacetone)palladium (0) in 20 ml of toluene, followed by addition of 1.58 g (16.5 mmol) of sodium tert-butoxide. After stirring for 20 hours at room temperature, the reaction medium is taken up in water and extracted with ethyl ether. The organic phase is washed with water, dried over Na₂SO₄ and concentrated under vacuum. The residue obtained is purified by flash chromatography on silica in a (1:1) heptane/ethyl acetate mixture to give 1.96 g of a beige-coloured solid, the NMR characteristics of which are identical to those of the product of Example 3a.

Yield: 65.8%

### b) 4-[(methoxyphenyl)amino]benzoic acid

Obtained by working as in Example 1b.

### c) 4-[(4-methoxyphenyl)(nitroso)amino]benzoic acid

Obtained by working as in Example 1c.

### Example 5

### 4-{Nitroso[4-(trifluoromethyl)phenyl]amino}benzoic acid

### a) ethyl 4-{[4-(trifluoromethyl)phenyl]amino}benzoate

A mixture composed of 9.1 g (28 mmol) of caesium carbonate, 0.458 g (0.5 mmol) of tris(dibenzylideneacetone)dipalladium, 0.934 g (1.5 mmol) of racemic BINAP (2,2-bis(diphenylphosphino)-1,1-binaphthyl), 4.5 g (20 mmol) of 1-bromo-4-(trifluoromethyl)benzene, 3.96 g (28 mmol) of ethyl 4-aminobenzoate and 60 ml of diglyme (diethylene glycol dimethyl ether) is heated for 6 hours at 100° C. After cooling, the reaction medium is poured into water and extracted with ether. The organic phase is washed with water, dried over Na₂SO₄ and concentrated under vacuum. The residue obtained is purified by flash chromatography on silica in an (11/9) dichloromethane/hexane mixture to give 5.5 g of a pale yellow solid.

Yield: 89%

IR (KBr): ν (NH): 3334 cm⁻¹; (CO): 1682 cm⁻¹, 1699 cm⁻¹

NMR: (CDCl₃): 1.35 (3H, t, J=7.1 Hz); 4.3 (2H, q, J=7.1 Hz); 6.15 (1H, broad s, exchangeable with D₂O); 7.0 (2H, m); 7.1 (2H, m); 7.5 (2H, m); 7.9 (2H, m)

### b) 4-{[4-(trifluoromethyl)phenyl]amino}benzoic acid

Obtained by working as in Example 1b.

Yield: 76.6%

IR (KBr): ν (NH): 3415 cm⁻¹; (CO): 1670 cm⁻¹

NMR: (DMSO-d6): 7.4 (2H, m); 7.5 (2H, m); 7.8 (2H, m); 8.0 (2H, m); 9.35 (1H, s, exchangeable with CF₃COOD); 12.7 (1H, broad s, exchangeable with CF₃COOD).

### c) 4-{nitroso[4-(trifluoromethyl)phenyl]amino}benzoic acid

Obtained by working as in Example 1c.

Yield: 81.7%

NMR: (DMSO-d6): 7.3 (1H, m); 7.5 (2H, m); 7.6 (1H, m); 7.8 (1H, m); 7.95 (1H, m); 8.1 (2H, m); 13.2 (1H, broad s)

### Example 6

### 4-[(4-Methoxyphenyl)(nitroso)amino]benzonitrile

### a) 4-[(4-methoxyphenyl)amino]benzonitrile

1.7 g (15 mmol) of sodium tert-butoxide are added to a mixture composed of 1.21 g (10 mmol) of 4-fluorobenzonitrile, 1.23 g (10 mmol) of 4-methoxyaniline and 10 ml of DMSO. The reaction medium is stirred for 24 hours at room temperature, and then poured into 120 ml of water and extracted with ether. The organic phase is washed with water until neutral, dried over Na₂SO₄ and concentrated under vacuum. The residue obtained is purified by flash chromatography on silica, in a (1/1) heptane/dichloromethane mixture to give 0.882 g of a pale yellow solid.

Yield: 39%

NMR: (DMSO-d6): 3.55 (3H, s); 6.65-6.8 (4H, m); 6.9 (2H, m); 7.3 (2H, m); 8.5 (1H, s)

This compound was also obtained by working as in Example 2a, in a yield of 61.9%.

### b) 4-[(4-methoxyphenyl)(nitroso)amino]benzonitrile

Obtained by working as in Example 1c.

Beige-coloured solid.

Yield: 83.4%

m.p.=88-90° C.

NMR: (DMSO-d6): 3.9 (3H, 2s); 7.1 (4H, m); 7.6 (2H, m); 8.0 (2H, m)

### Example 7

### 4-[(4-Methoxyphenyl)(nitroso)amino]benzoic acid

### a) 4-[(4-methoxyphenyl)amino]benzoic acid

A mixture of 3 g (13.4 mmol) of 4-[(4-methoxyphenyl)amino]benzonitrile obtained in Example 6a, 1.5 g (26.8 mmol) of KOH and 80 ml of ethylene glycol is refluxed for 4 hours. After cooling, the mixture is poured into ice-cold water and acidified with acetic acid. The precipitate formed is filtered off by suction, washed with water and dried under vacuum. 2.9 g of a beige-coloured solid, which has the same spectral (IR, NMR) properties as the compound obtained in Example 1b, are obtained.

Yield: 89.2%

### b) 4-[(4-methoxyphenyl)(nitroso)amino]benzoic acid

Obtained by working as in Example 1c.

### Example 8

### {4-[(4-Methoxyphenyl)(nitroso)amino]phenyl}methanol

### a) {4-[(4-methoxyphenyl)amino]phenyl}methanol

A solution of 1 g (3.7 mmol) of ethyl 4-[(4-methoxyphenyl)amino]benzoate, obtained as in Example 3a, in 10 ml of THF is added dropwise to a suspension of 0.21 g (5.5 mmol) of LiAlH₄ in 15 ml of THF. The reaction medium is then refluxed for 2 hours. After cooling, 1 ml of ethyl acetate is added dropwise, the resulting mixture is then hydrolysed by dropwise addition of water, and finally 20 ml of ether are added. The precipitate formed is filtered off and rinsed with ether. The filtrate is concentrated under vacuum and the residue obtained is purified by flash chromatography on silica in a (4/1) and then (1/1) heptane/ethyl acetate mixture, to give 0.34 g of a pink solid.

Yield: 40.3%

m.p.=110° C.

NMR: (DMSO-d6): 3.9 (3H, s); 4.57 (2H, d, J=5.6 Hz); 5.15 (1H, t, J=5.6 Hz); 7.05 (4H, m); 7.20 (2H, m); 7.25 (2H, m); 7.95 (1H, s).

### b) {4-[(4-methoxyphenyl)(nitroso)amino]phenyl}methanol

Obtained by working as in Example 1c, after purification by flash chromatography on silica in a (4/1) dichloromethane/ether mixture. Red oil.

Yield: 63.9%

NMR: (DMSO-d6): 3.80 (3H, 2s); 4.54 (2H, 2d, J=5.9 Hz, transforms 2s with CF₃COOD); 5.30 (1H, 2t, J=5.9 Hz, exchangeable with CF₃COOD); 6.9 (4H, m); 7.15-7.6 (4H, m)

### Examples 9 to 29

The compounds of Examples 9a to 29a and 9b to 29b were obtained as in Example 1.

Their structure and characteristics are collated in Tables 1 and 2, respectively.

TABLE 1

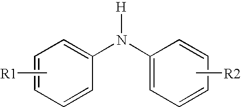

| Ex | | R1 | R2 | NMR |
|---|---|---|---|---|
| 9a | 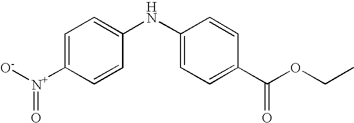 | 4-NO₂ | 4-CO₂Et | (CDCl₃): 1.36 (3H, t, J=7.2 Hz); 4.35 (2H, q, J=7.2 Hz); 6.47 (1H, broad s); 6.99-7.32 (4H, m); 7.92-8.30 (4H, m). |
| 10a | 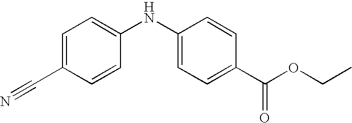 | 4-CN | 4-CO₂Et | (CDCl₃): 1.39 (3H, t, J=7.2 Hz); 4.35 (2H, q, J=7.2 Hz); 6.29 (1H, broad s); 7.02-7.20 (4H, m); 7.44-7.67 (2H, m); 7.88-8.14 (2H, m). |
| 11a | 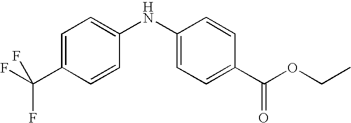 | 4-CF₃ | 4-CO₂Et | (CDCl₃): 1.36 (3H, t, J=7.2 Hz); 4.33 (2H, q, J=7.2 Hz); 6.22 (1H, broad s); 7.00-7.22 (4H, m); 7.44-7.63 (2H, m); 7.82-8.11 (2H, m). |
| 12a | 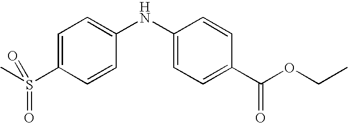 | 4-MeSO₂ | 4-CO₂Et | (CDCl₃): 1.39 (3H, t, J=7.2 Hz); 4.36 (2H, q, J=7.2 Hz); 3.04 (3H, s); 6.39 (1H, s); 7.05-7.38 (4H, m); 7.67-8.20 (4H, m). |
| 13a | 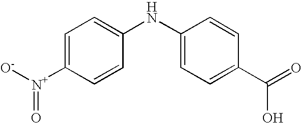 | 4-NO₂ | 4-CO₂H | (DMSO-d6): 7.06-7.46 (4H, m); 7.81-8.28 (4H, m); 9.62 (1H, broad s). |
| 14a | 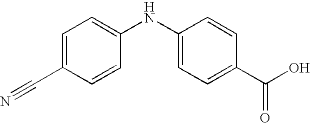 | 4-CN | 4-CO₂H | (DMSO-d6): 6.92-7.44 (4H, m); 7.48-8.07 (4H, m); 9.30 (1H, broad s); 12.59 (1H, broad s). |
| 15a | 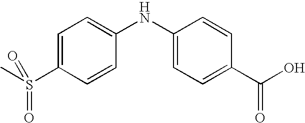 | 4-MeSO₂ | 4-CO₂H | (DMSO-d6): 3.13 (3H, s); 7.05-7.55 (4H, m); 7.55-8.11 (4H, m); 9.29 (1H, s); 12.51 (1H, broad s). |
| 16a | 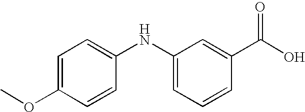 | 4-MeO | 3-CO₂H | (DMSO-d6): 3.72 (3H, s); 6.82-6.95 (2H, m); 6.98-7.16 (3H, m); 7.18-7.30 (2H, m); 7.39-7.52 (1H, m); 8.04 (1H, s); 12.73 (1H, broad s). |
| 17a | 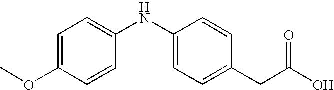 | 4-MeO | 4-CH₂—CO₂H | (DMSO-d6): 3.40 (2H, s); 3.70 (3H, s); 6.75-6.93 (4H, m); 6.93-7.14 (4H, m); 7.78 (1H, s); 12.16 (1H, broad s). |
| 18a | 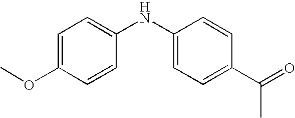 | 4-MeO | 4-COMe | (DMSO-d6): 2.41 (3H, s); 3.73 (3H, s); 6.75-7.04 (4H, m); 7.04-7.22 (2H, m); 7.63-7.99 (2H, m); 8.55 (1H, s). |
| 19a | 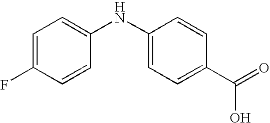 | 4-F | 4-COOH | (DMSO-d6): 6.66-7.46 (6H, m); 7.55-8.03 (2H, m); 8.65 (1H, s); 12.29 (1H, broad s). |
| 20a | 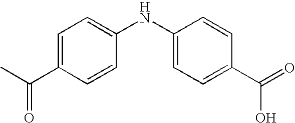 | 4-MeCO | 4-COOH | (DMSO-d6): 2.49 (3H, s); 6.91-7.46 (4H, m); 7.64-8.01 (4H, m); 9.23 (1H, s); 12.50 (1H, broad s). |
| 21a | 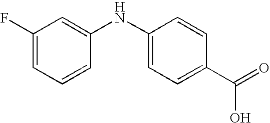 | 3-F | 4-COOH | (DMSO-d6): 6.59-7.46 (6H, m); 7.68-7.97 (2H, m); 8.90 (1H, s); 12.39 (1H, broad s). |
| 22a | 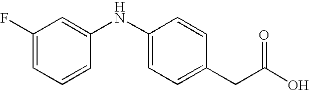 | 3-F | 4-CH₂—CO2H | (DMSO-d6): 3.47 (2H, s); 6.33-6.93 (3H, m); 6.93-7.34 (5H, m); 8.35 (1H, s); 12.24 (1H, broad s). |
| 23a | 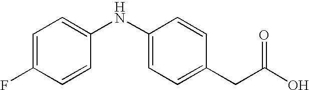 | 4-F | 4-CH₂—CO2H | (DMSO-d6): 3.43 (2H, s); 6.78-7.22 (8H, m); 8.04 (1H, s); 12.20 (1H, broad s). |
| 24a | 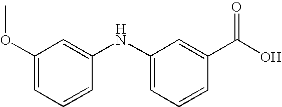 | 3-MeO | 3-CO₂H | (DMSO-d6): 3.71 (3H, s); 6.30-6.85 (3H, m); 6.96-7.54 (4H, m); 7.54-7.73 (1H, m); 8.37 (1H, s); 12.83 (1H, broad s). |
| 25a | 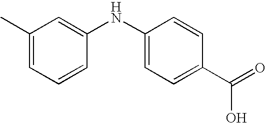 | 3-Me | 4-CO₂H | (DMSO-d6): 2.27 (3H, s); 6.50-7.48 (6H, m); 7.57-7.98 (2H, m); 8.63 (1H, s); 12.83 (1H, broad s). |
| 26a | 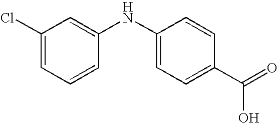 | 3-Cl | 4-CO₂H | (DMSO-d6): 6.77-7.52 (6H, m); 7.52-8.21 (2H, m); 8.87 (1H, s); 12.40 (1H, broad s). |
| 27a | 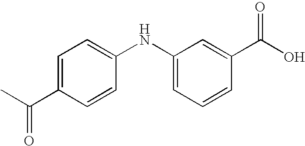 | 4-MeCO | 3-CO₂H | (DMSO-d6): 2.49 (3H, s); 6.80-8.17 (8H, m); 9.01 (1H, s); 12.99 (1H, broad s). |
| 28a | 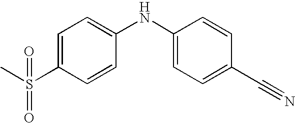 | 4-MeSO₂ | 4-CN | (DMSO-d6): 3.14 (3H, s); 7.09-7.46 (4H, m); 7.54-7.91 (4H, m); 9.43 (1H, s). |
| 29a | 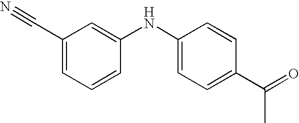 | 3-CN | 4-COMe | (DMSO-d6): 2.49 (3H, s); 7.05-7.63 (6H, m); 7.79-7.99 (2H, m); 9.11 (1H, s). |

TABLE 2

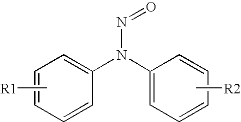

| Ex | | R1 | R2 | NMR |
|---|---|---|---|---|
| 9b | 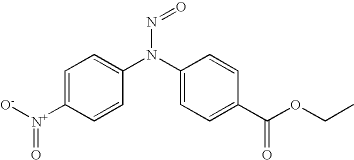 | 4-NO₂ | 4-CO₂Et | (DMSO-d6): 1.03-1.58 (3H, m); 4.11-4.56 (2H, m); 7.18-7.86 (4H, m); 7.86-8.65 (4H, m). |
| 10b | 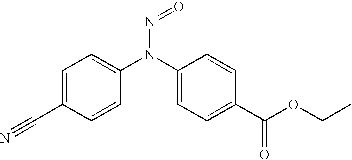 | 4-CN | 4-CO₂Et | (DMSO-d6): 1.21-1.50 (3H, m); 4.16-4.52 (2H, m); 7.22-7.73 (4H, m); 7.81-8.28 (4H, m). |
| 11b | 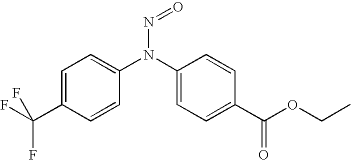 | 4-CF₃ | 4-CO₂Et | (DMSO-d6): 1.13-1.49 (3H, m); 4.16-4.49 (2H, m); 7.26-7.75 (4H, m); 7.75-8.29 (4H, m). |
| 12b | 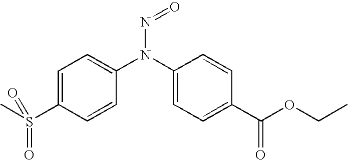 | 4-MeSO₂ | 4-CO₂Et | (DMSO-d6): 1.04-1.53 (3H, m); 3.31 and 3.35 (3H, 2s); 4.11-4.59 (2H, m); 7.07-8.43 (8H, m). |
| 13b | 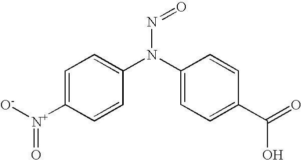 | 4-NO₂ | 4-CO₂H | (DMSO-d6): 7.05-7.79 (4H, m); 7.80-8.70 (4H, m); 13.34 (1H, broad s). |
| 14b | 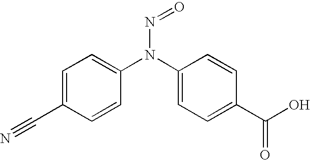 | 4-CN | 4-CO₂H | (DMSO-d6): 7.09-7.77 (4H, m); 7.77-8.36 (4H, m); 13.08 (1H, broad s). |
| 15b | 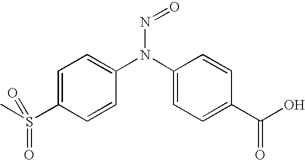 | 4-MeSO₂ | 4-CO₂H | (DMSO-d6): 3.25 and 3.32 (3H, 2s); 7.21-7.82 (4H, m); 7.82-7.45 (4H, m); 12.93 (1H, broad s). |
| 16b | 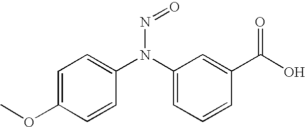 | 4-MeO | 3-CO₂H | (DMSO-d6): 3.79 and 3.82 (3H, 2s); 6.73-8.29 (8H, m); 13.19 (1H, broad s). |
| 17b | 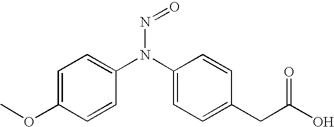 | 4-MeO | 4-CH₂—CO₂H | (DMSO-d6): 3.61 and 3.65 (2H, 2s); 3.78 and 3.80 (3H, 2s); 6.80-7.61 (8H, m); 12.35 (1H, broad s). |
| 18b | 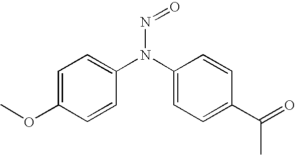 | 4-MeO | 4-COMe | (DMSO-d6): 2.58 and 2.61 (3H, 2s); 3.79 and 3.82 (3H, 2s); 6.91-8.38 (8H, m). |
| 19b | 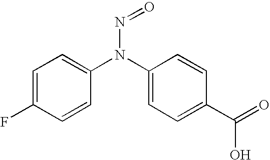 | 4-F | 4-COOH | (DMSO-d6): 7.00-7.78 (6H, m); 7.78-8.35 (2H, m); 13.10 (1H, broad s). |
| 20b | 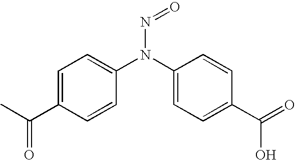 | 4-MeCO | 4-COOH | (DMSO-d6): 2.58 and 2.63 (3H, 2s); 6.97-7.82 (5H, m); 7.82-8.55 (3H, m); 13.19 (1H, broad s). |
| 21b | 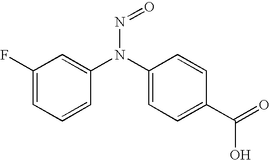 | 3-F | 4-COOH | (DMSO-d6): 6.86-7.87 (6H, m); 7.87-8.30 (2H, m); 13.15 (1H, s). |
| 22b | 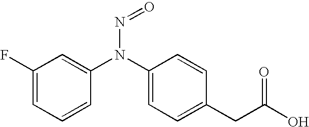 | 3-F | 4-CH₂—CO2H | (DMSO-d6): 3.63 and 3.67 (2H, 2s); 6.79-8.07 (8H, m); 12.44 (1H, s). |
| 23b | 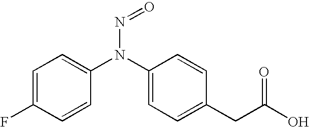 | 4-F | 4-CH₂—CO2H | (DMSO-d6): 3.62 and 3.66 (2H, 2s); 6.77-7.91 (8H, m); 12.42 (1H, broad s). |
| 24b | 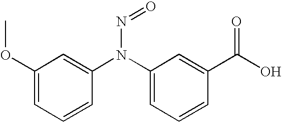 | 3-MeO | 3-CO₂H | (DMSO-d6): 3.76 and 3.78 (3H, 2s); 6.54-8.19 (8H, m); 13.31 (1H, broad s). |
| 25b | 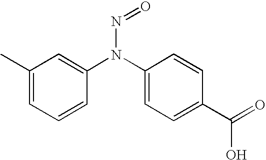 | 3-Me | 4-CO₂H | (DMSO-d6): 2.34 (3H, s); 6.88-7.73 (6H, m); 7.75-8.32 (2H, m); 13.13 (1H, broad s). |
| 26b | 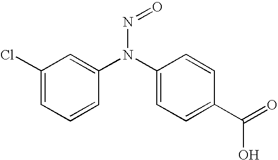 | 3-Cl | 4-CO₂H | (DMSO-d6): 6.97-7.84 (6H, m); 7.84-8.35 (2H, m); 13.14 (1H, m). |
| 27b | 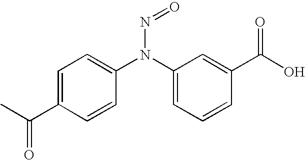 | 4-MeCO | 3-CO₂H | (DMSO-d6): 2.59 and 2.63 (3H, 2s); 6.90-8.46 (8H, m); 13.30 (1H, m). |
| 28b | 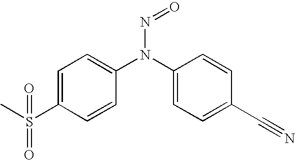 | 4-MeSO₂ | 4-CN | (DMSO-d6): 3.25 and 3.32 (3H, 2s); 7.32-7.82 (4H, m); 7.82-8.42 (4H, m). |
| 29b | 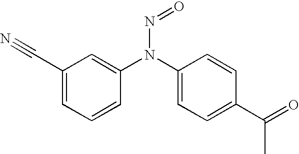 | 3-CN | 4-COMe | (DMSO-d6): 2.59 and 2.63 (3H, 2s); 7.07-8.46 (8H, m). |

### Example 30

### 4,4′-(Nitrosoimino)dibenzoic acid

### a) 4,4′-iminodibenzoic acid

Obtained by working as in Example 7a, starting with 4-[(4-cyanophenyl)amino]benzoic acid (Example 14a).

Yield: 89.4%

IR (KBr): ν (NH): 3404 cm⁻¹; (CO): 1667 cm⁻¹

NMR: (DMSO-d6): 7.2 (4H, m); 7.85 (4H, m); 9.2 (1H, s, exchangeable with D₂O); 12.5 (1H, broad s, exchangeable with CF₃COOD)

### b) 4,4′-(nitrosoimino)dibenzoic acid

Obtained by working as in Example 1c.

Beige-coloured solid.

Yield: 88.2%

IR (KBr): ν (CO): 1688 cm⁻¹

NMR: (DMSO-d6): 7.45 (2H, m); 7.65 (2H, m); 8.1 (2H, m); 8.2 (2H, m); 13.3 (1H, broad s)

### Examples 31 to 52

The compounds of Examples 31a to 52a were obtained as in Example 1a-1b, 5a-5b or 8a. The compounds of Examples 31b to 52b were obtained as in Example 1c. Their structure and characteristics are collated in Tables 3 and 4, respectively. The NMR spectra of Tables 3 and 4 were acquired in DMSO-d₆.

TABLE 3

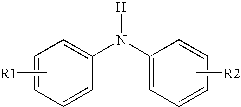

| Ex | | R1 | R2 | NMR |
|---|---|---|---|---|
| 31a | 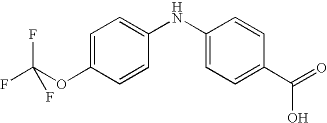 | 4-F₃CO | 4-CO₂H | 7.07 (d, J=8.77 Hz, 2H) 7.25 (m, 4H) 7.79 (d, J=8.77 Hz, 2H) 8.86 (s, 1H) 12.35 (s, 1H) |
| 32a | 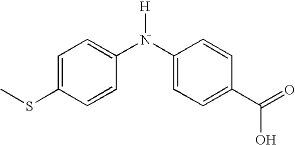 | 4-MeS | 4-CO₂H | 2.44 (s, 3H) 7.14 (m, 6H) 7.76 (d, J=8.58 Hz, 2H) 8.70 (s, 1H) 12.29 (s, 1H) |
| 33a | 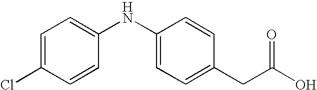 | 4-Cl | 4-CH₂—CO₂H | 3.46 (s, 2H) 7.09 (m, 8H) 8.24 (s, 1H) 12.20 (s, 1H) |
| 34a | 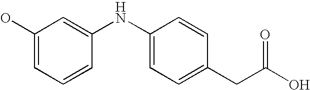 | 3-MeO | 4-CH₂—CO₂H | 3.45 (s, 2H) 3.69 (s, 3H) 6.37 (dd, J=8.20, 1.72 Hz, 1H) 6.59 (m, 2H) 7.06 (m, 5H), 8.11 (s, 1H) 12.21 (s, 1H) |
| 35a | 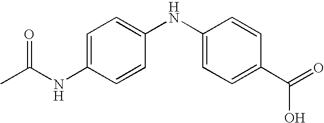 | 4-AcNH | 4-CO₂H | 2.01 (s, 3H) 6.94 (d, J=8.77 Hz, 2H) 7.10 (d, J=8.77 Hz, 2H) 7.51 (d, J=8.77 Hz, 2H) 7.74 (d, J=8.77 Hz, 2H) 8.57 (s, 1H) 9.85 (s, 1H) 12.22 (s, 1H) |
| 36a | 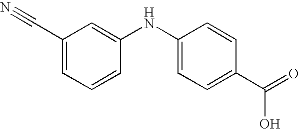 | 3-CN | 4-CO₂H | 7.34 (m, 6H) 7.83 (d, J=8.58 Hz, 2H) 9.01 (s, 1H) 12.46 (s, 1H) |
| 37a | 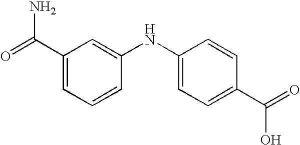 | 3-CN | 4-CO₂H | 3.54 (s, 3H) 7.25 (m, 5H) 7.75 (m, 3H) 8.65 (s, 1H) |
| 38a | 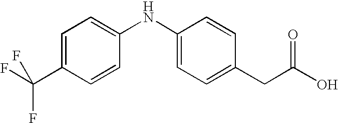 | 4-CF₃ | 4-CH₂—CO₂H | 3.49 (s, 2H) 7.14 (m, 6H) 7.48 (d, J=8.58 Hz, 2H) 8.63 (s, 1H) 12.28 (s, 1H) |
| 39a | 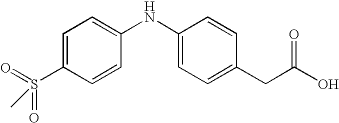 | 4-MeSO₂ | 4-CH₂—CO₂H | 3.08 (s, 3H) 3.51 (s, 2H) 7.14 (m, 6H) 7.67 (d, J=8.77 Hz, 2H) 8.83 (s, 1H) 12.29 (s, 1H) |
| 40a | 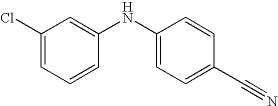 | 3-Cl | 4-CN | 7.14 (m, 6H) 7.63 (m, 2H) 9.05 (s, 1H) |
| 41a | 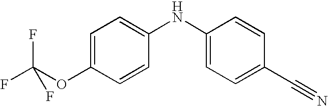 | 4-CF₃O | 4-CN | 7.24 (m, 6H) 7.61 (d, J=8.58 Hz, 2H) 9.03 (s, 1H) |
| 42a | 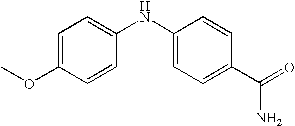 | 4-MeO | 4-CONH₂ | 3.72 (s, 3H) 6.87 (m, 5H) 7.09 (d, J=8.67 Hz, 2H) 7.61 (s, 1H) 7.68 (d, J=8.29 Hz, 2H) 8.24 (s, 1H) |
| 43a | 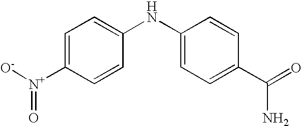 | 4-NO₂ | 4-CONH₂ | 7.20 (m, 5H) 7.86 (m, 3H) 8.12 (m, 2H) 9.51 (s, 1H) |
| 44a | 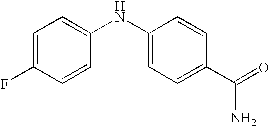 | 4-F | 4-CONH₂ | 7.08 (m, 7H) 7.73 (m, 3H) 8.47 (s, 1H) |
| 45a | 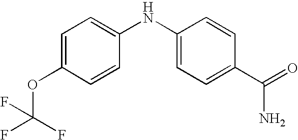 | 4-CF₃O | 4-CONH₂ | 7.17 (m, 7H) 7.75 (m, 3H) 8.70 (s, 1H) |
| 46a | 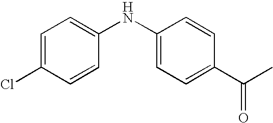 | 4-Cl | 4-COMe | 2.45 (s, 3H) 7.22 (m, 6H) 7.83 (d, J=8.77 Hz, 2H) 8.90 (s, 1H) |
| 47a | 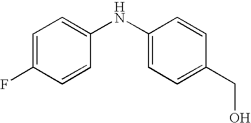 | 4-F | 4-CH₂OH | 4.37 (d, J=5.53 Hz, 2H) 4.97 (m, 1H) 7.05 (m, 8H) 8.02 (s, 1H) |
| 48a | 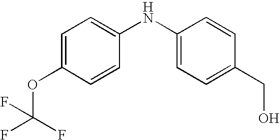 | 4-CF₃O | 4-CH₂OH | 4.40 (d, J=5.53 Hz, 2H) 5.01 (m, 1H) 7.11 (m, 8H) 8.28 (s, 1H) |
| 49a | 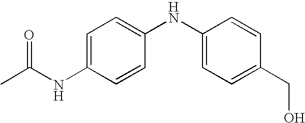 | 4-NHAc | 4-CH₂OH | 3.32 (s, 3H) 4.36 (d, J=5.53 Hz, 2H) 4.94 (d, J=5.53 Hz, 1H) 7.00 (m, 6H) 7.40 (m, 2H) 7.93 (s, 1H) 9.72 (s, 1H) |
| 50a | 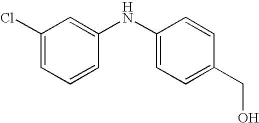 | 3-Cl | 4-CH₂OH | 4.41 (d, J=5.53 Hz, 2H) 5.03 (t, J=5.53 Hz, 1H) 6.77 (m, 1H) 7.08 (m, 7H) 8.32 (s, 1H) |
| 51a | 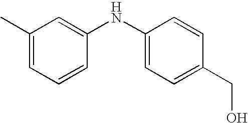 | 3-Me | 4-CH₂OH | 2.22 (s, 3H) 4.38 (d, J=5.53 Hz, 2H) 4.97 (t, J=5.63 Hz, 1H) 6.60 (d, J=7.25 Hz, 1H) 7.02 (m, 7H) 7.99 (s, 1H) |
| 52a | 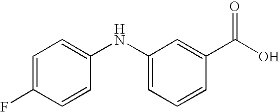 | 4-F | 3-CO₂H | 7.31 (m, 8H) 8.38 (s, 1H) 12.90 (s, 1H) |

TABLE 4

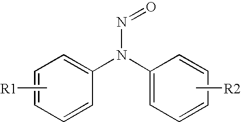

| Ex | | R1 | R2 | NMR |
|---|---|---|---|---|
| 31b | 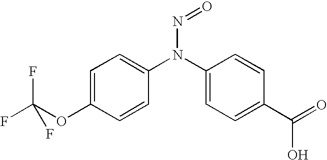 | 4-F₃CO | 4-CO₂H | 7.47 (m, 6H) 8.07 (m, 2H) 13.17 (s, 1H) |
| 32b | 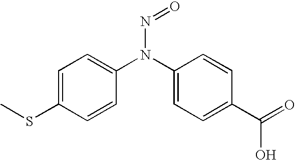 | 4-MeS | 4-CO₂H | 2.52 (s, 3H) 7.12 (d, J=8.58 Hz, 2H) 7.40 (m, 4H) 8.06 (m, 2H) 13.11 (s, 1H) |
| 33b | 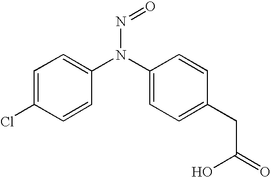 | 4-Cl | 4-CH₂—CO₂H | 3.65 (2s, 2H) 7.32 (m, 6H) 7.59 (dd, J=18.79, 8.87 Hz, 2H) 12.44 (s, 1H) |
| 34b | 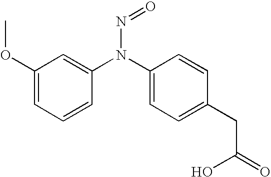 | 3-MeO | 4-CH₂—CO₂H | 3.64 (2s, 2H) 3.76 (2s, 3H) 6.90 (m, 4H) 7.43 (m, 4H) 12.42 (s, 1H) |
| 35b | 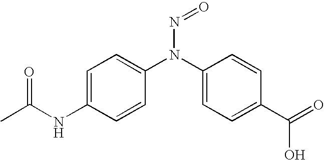 | 4-AcNH | 4-CO₂H | 2.06 (2s, 3H) 7.45 (m, 6H) 8.03 (d, J=8.77 Hz, 2H) 10.21 (s, 1H) 13.11 (s, 1H) |
| 36b | 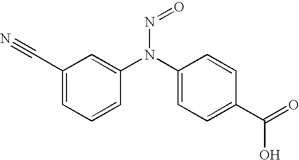 | 3-CN | 4-CO₂H) | 7.78 (m, 8H) 13.16 (s, 1H) |
| 37b | 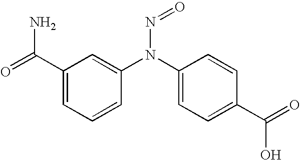 | 3-CONH₂ | 4-CO₂H | 7.74 (m, 10H) 13.08 (s, 1H) |
| 38b | 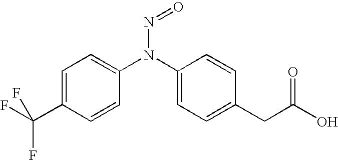 | 4-CF₃ | 4-CH₂—CO₂H | 3.68 (2s, 2H) 7.37 (m, 6H) 7.86 (d, J=8.58 Hz, 2H) 12.53 (s, 1H) |
| 39b | 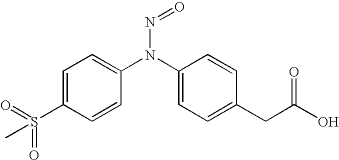 | 4-MeSO₂ | 4-CH₂—CO₂H | 3.28 (2s, 3H) 3.67 (2s, 2H) 7.42 (m, 6H) 8.06 (m, 2H) 12.46 (s, 1H) |
| 40b | 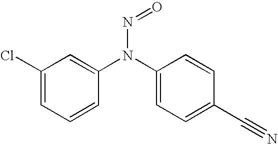 | 3-Cl | 4-CN | 7.22 (m, 1H) 7.56 (m, 5H) 8.00 (m, 2H) |
| 41b | 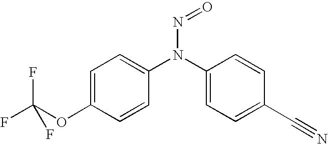 | 4-CF₃O | 4-CN | 7.50 (m, 6H) 8.00 (m, 2H) |
| 42b | 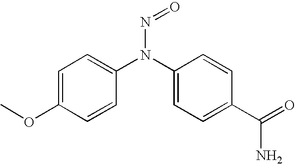 | 4-MeO | 4-CONH₂ | 3.80 (dd, J=6.97, 3.96 Hz, 3H) 7.29 (m, 7H) 7.97 (m, 3H) |
| 43b | 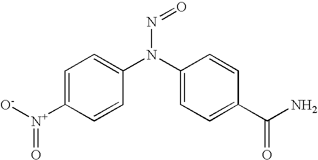 | 4-NO₂ | 4-CONH₂ | 7.53 (m, 5H) 8.26 (m, 5H) |
| 44b | 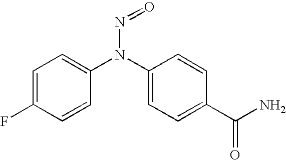 | 4-F | 4-CONH₂ | 7.36 (m, 7H) 7.99 (m, 3H) |
| 45b | 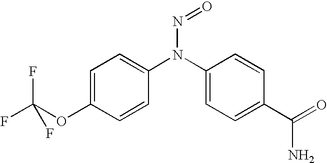 | 4-CF₃O | 4-CONH₂ | 7.44 (m, 7H) 8.01 (m, 3H) |
| 46b | 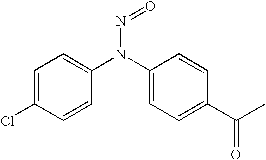 | 4-Cl | 4-COMe | 2.61 (2s, 3H) 7.47 (m, 6H) 8.08 (m, 2H) |
| 47b | 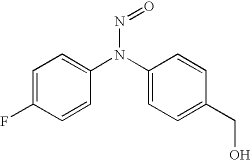 | 4-F | 4-CH₂OH | 4.54 (m, 2H) 5.29 (m, 1H) 7.33 (m, 8H) |
| 48b | 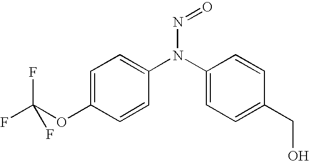 | 4-CF3O | 4-CH₂OH | 4.55 (dd, J=8.87, 5.63 Hz, 2H) 5.33 (m, 1H) 7.38 (m, 8H) |
| 49b | 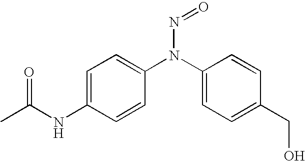 | 4-NHAc | 4-CH₂OH | 2.12 (2s, 3H) 4.58 (d, J=8.39 Hz, 2H) 5.33 (d, J=3.62 Hz, 1H) 7.47 (m, 8H) 10.25 (m, 1H) |
| 50b | 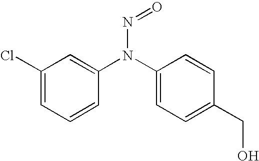 | 3-Cl | 4-CH₂OH | 4.55 (dd, J=9.63, 5.82 Hz, 2H) 5.32 (m, 1H) 7.38 (m, 8H) |
| 51b | 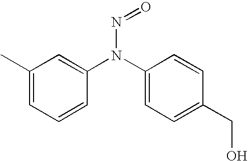 | 3-Me | 4-CH₂OH | 2.33 (s, 3H) 4.54 (dd, J=10.20, 5.44 Hz, 2H) 5.30 (m, 1H) 7.27 (m, 8H) |
| 52b | 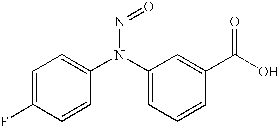 | 4-F | 3-CO₂H | 7.47 (m, 8H) 13.33 (s, 1H) |

1. Compounds of the formula I 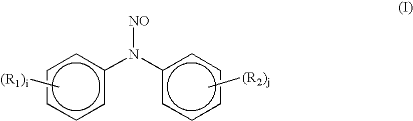
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an alkylcarbonyl group; a carboxyl group; an alkylcarbonylamino group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,
with the exclusion of the compound for which i and j=1 and R₁=carboxyl and R₂=alkoxycarbonyl or R₁=CF₃ and R₂=carboxyl; and also the pharmaceutically acceptable derivatives, salts, solvates and stereoisomers thereof, including mixtures thereof in all proportions.

2. Compounds according to formula (I) of claim 1, in which the aliphatic hydrocarbon-based group is a linear or branched C₂-C₁₀ alkyl group optionally substituted by halogen, nitro, cyano, amino, mono- or dialkylamino, carboxyl, acylamino or alkylsulfonyl.

3. Compounds according to formula (a) of claim 1, in which the halogen is fluorine.

4. Compounds according to formula (I) of claim 1, in which R₁ represents, independently of each other, —OCH₃, —SCH₃, —OCF₃, —N(CH₃)₂, —COCH₃ or methylsulfonyl.

5. Compounds of the formula (I) according to claim 1, in which R₂ represents, independently of each other, cyano, —CH₂OH, —COCH₃, —COOH, —CH₂COOH, —COOCH₃, —COOC₂H₅ or CONH₂.

6. Compounds of the formula (I) according to claim 1, in which i=1.

7. Compounds of the formula (I) according to claim 1 in which j=1.

8. Compounds of the formula (I) according to claim 1, in which at least one R₁ is in position 3 or 4 on the phenyl ring.

9. Compounds of the formula (I) according to claim 1, in which at least one R₂ is in position 3 or 4 on the phenyl ring.

10. Compounds of the formula (I) according to claim 1, chosen from the following compounds:
4-[(4-methoxyphenyl)(nitroso)amino]benzoic acid;
4-{nitroso[4-(trifluoromethyl)phenyl]amino}benzoic acid;
4-[(4-methoxyphenyl)(nitroso)amino]benzonitrile;
{4-[(4-methoxyphenyl)(nitroso)amino]phenyl}methanol;
Ex. 9-29;
N-nitrosodiphenylamine-4,4′-dicarboxylic acid;
4-{nitroso[4-(trifluoromethoxy)phenyl]amino}benzoic acid;
4-[[4-(methylthio)phenyl](nitroso)amino]benzoic acid;
{4-[(4-chlorophenyl)(nitroso)amino]phenyl}acetic acid;
{4-[(3-methoxyphenyl)(nitroso)amino]phenyl}acetic acid;
4-[[4-(acetylamino)phenyl](nitroso)amino]benzoic acid;
4-[(3-cyanophenyl)(nitroso)amino]benzoic acid;
4-[[3-(aminocarbonyl)phenyl](nitroso)amino]benzoic acid;
(4-{nitroso[4-(trifluoromethyl)phenyl]amino}phenyl)acetic acid;
{4-[[4-(methylsulfonyl)phenyl](nitroso)amino]phenyl}acetic acid;
4-[(3-chlorophenyl)(nitroso)amino]benzonitrile;
4-{nitroso[4-(trifluoromethoxy)phenyl]amino}benzonitrile;
4-[(4-methoxyphenyl)(nitroso)amino]benzamide;
4-[(4-nitrophenyl)(nitroso)amino]benzamide;
4-[(4-fluorophenyl)(nitroso)amino]benzamide;
4-{nitroso[4-(trifluoromethoxy)phenyl]amino}benzamide;
1-{4-[(4-chlorophenyl)(nitroso)amino]phenyl}ethanone;
{4-[(4-fluorophenyl)(nitroso)amino]phenyl}methanol;
(4-{nitroso[4-(trifluoromethoxy)phenyl]amino}phenyl)methanol;
N-{4-[[4-(hydroxymethyl)phenyl](nitroso)amino]phenyl}acetamide;
{4-[(3-chlorophenyl)(nitroso)amino]phenyl}methanol;
{4-[(3-methylphenyl)(nitroso)amino]phenyl}methanol;
3-[(4-fluorophenyl)(nitroso)amino]benzoic acid.

11. Process for the preparation of a compound of the formula (I) according to claim 1, comprising the reaction of a compound of the formula (II): 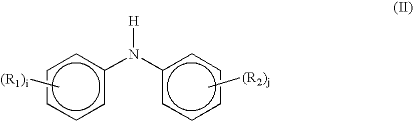
in which R₁, R₂, i and j are as defined for formula (I) in claim 1, with a nitrosating agent.

12. Pharmaceutical composition comprising a compound of the formula (I) 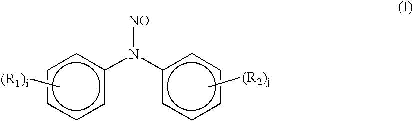
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an acylamino group, an alkylcarbonyl group; a carboxyl group; an unsubstituted amide group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,
with the exclusion of the compound for which i and j=1 and R₁=carboxyl and R₂=alkoxycarbonyl or R₁=CF₃ and R₂=carboxyl;
and also the pharmaceutically acceptable derivatives, salts, solvates and stereoisomers thereof, including mixtures thereof in all proportions.

13. Composition according to claim 12, in which the aliphatic hydrocarbon-based group is a linear or branched C₂-C₆ alkyl group optionally substituted by halogen, nitro, cyano, amino, mono- or dialkylamino, carboxyl, unsubstituted amide; alkylsulfonyl.

14. Composition according to claim 12, in which the halogen is fluorine.

15. Composition according to claim 13, in which R₁ represents, independently of each other, —OCH₃, —SCH₃, —OCF₃, —N(CH₃)₂, —COCH₃, methylsulfonyl.

16. Composition according to claim 13, in which R₂ represents, independently of each other, cyano, —CH₂OH, —COCH₃, —COOH, —CH₂COOH, —COOCH₃, —COOC₂H₅ or unsubstituted amide.

17. Composition according to claim 13, in which i=1.

18. Composition according to claim 13, in which j=1.

19. Composition according to claim 13, in which at least one R₁ is in position 3 or 4 on the phenyl ring.

20. Composition according to claim 13, in which at least one R₂ is in position 3 or 4 on the phenyl ring.

21. Composition according to claim 13, in which the compound of the formula (I) is chosen from the following compounds:
4-[(4-methoxyphenyl)(nitroso)amino]benzoic acid;
4-{nitroso[4-(trifluoromethyl)phenyl]amino}benzoic acid;
4-[(4-methoxyphenyl)(nitroso)amino]benzonitrile;
{4-[(4-methoxyphenyl)(nitroso)amino]phenyl}methanol;
ethyl 4-[(4-nitrophenyl)(nitroso)amino]benzoate;
ethyl 4-[(4-cyanophenyl)(nitroso)amino]benzoate;
ethyl 4-{nitroso[4-(trifluoromethyl)phenyl]amino}benzoate;
ethyl 4-[[4-(methylsulfonyl)phenyl](nitroso)amino]benzoate;
4-[(4-nitrophenyl)(nitroso)amino]benzoic acid;
4-[(4-cyanophenyl)(nitroso)amino]benzoic acid;
4-[[4-(methylsulfonyl)phenyl](nitroso)amino]benzoic acid;
3-[(4-methoxyphenyl)(nitroso)amino]benzoic acid;
{4-[(4-methoxyphenyl)(nitroso)amino]phenyl}acetic acid;
1-{4-[(4-methoxyphenyl)(nitroso)amino]phenyl}ethanone;
4-[(4-fluorophenyl)(nitroso)amino]benzoic acid;
4-[(4-acetylphenyl)(nitroso)amino]benzoic acid;
4-[(3-fluorophenyl)(nitroso)amino]benzoic acid;
{4-[(3-fluorophenyl)(nitroso)amino]phenyl}acetic acid;
{4-[(4-fluorophenyl)(nitroso)amino]phenyl}acetic acid;
3-[(3-methoxyphenyl)(nitroso)amino]benzoic acid;
4-[(3-methylphenyl)(nitroso)amino]benzoic acid;
4-[(3-chlorophenyl)(nitroso)amino]benzoic acid;
3-[(4-acetylphenyl)(nitroso)amino]benzoic acid;
4-[[4-(methylsulfonyl)phenyl](nitroso)amino]benzonitrile;
3-[(4-acetylphenyl)(nitroso)amino]benzonitrile;
N-nitrosodiphenylamine-4,4′-dicarboxylic acid;
4-{nitroso[4-(trifluoromethoxy)phenyl]amino}benzoic acid;
4-[[4-(methylthio)phenyl](nitroso)amino]benzoic acid;
{4-[(4-chlorophenyl)(nitroso)amino]phenyl}acetic acid;
{4-[(3-methoxyphenyl)(nitroso)amino]phenyl}acetic acid;
4-[[4-(acetylamino)phenyl](nitroso)amino]benzoic acid;
4-[(3-cyanophenyl)(nitroso)amino]benzoic acid;
4-[[3-(aminocarbonyl)phenyl](nitroso)amino]benzoic acid;
(4-{nitroso[4-(trifluoromethyl)phenyl]amino}phenyl)acetic acid;
{4-[[4-(methylsulfonyl)phenyl](nitroso)amino]phenyl}acetic acid;
4-[(3-chlorophenyl)(nitroso)amino]benzonitrile;
4-{nitroso[4-(trifluoromethoxy)phenyl]amino}benzonitrile;
4-[(4-methoxyphenyl)(nitroso)amino]benzamide;
4-[(4-nitrophenyl)(nitroso)amino]benzamide;
4-[(4-fluorophenyl)(nitroso)amino]benzamide;
4-{nitroso[4-(trifluoromethoxy)phenyl]amino}benzamide;
1-{4-[(4-chlorophenyl)(nitroso)amino]phenyl}ethanone;
{4-[(4-fluorophenyl)(nitroso)amino]phenyl}methanol;
(4-{nitroso[4-(trifluoromethoxy)phenyl]amino}phenyl)methanol;
N-{4-[[4-(hydroxymethyl)phenyl](nitroso)amino]phenyl}acetamide;
{4-[(3-chlorophenyl)(nitroso)amino]phenyl}methanol;
{4-[(3-methylphenyl)(nitroso)amino]phenyl}methanol;
3-[(4-fluorophenyl)(nitroso)amino]benzoic acid.

22. Use of a compound of the formula (I) 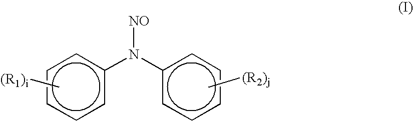
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an acylamino group, an alkylcarbonyl group; a carboxyl group; an unsubstituted amide group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group;
i and j independently being 1 to 5,
and also the pharmaceutically acceptable derivatives, salts, solvates and stereoisomers thereof, including mixtures thereof in all proportions.

23. Use according to claim 22, in which the compound of the formula (I) is chosen from the following compounds:
4-[(4-methoxyphenyl)(nitroso)amino]benzoic acid;
4-{nitroso[4-(trifluoromethyl)phenyl]amino}benzoic acid;
4-[(4-methoxyphenyl)(nitroso)amino]benzonitrile;
{4-[(4-methoxyphenyl)(nitroso)amino]phenyl}methanol;
ethyl 4-[(4-nitrophenyl)(nitroso)amino]benzoate;
ethyl 4-[(4-cyanophenyl)(nitroso)amino]benzoate;
ethyl 4-{nitroso[4-(trifluoromethyl)phenyl]amino}benzoate;
ethyl 4-[[4-(methylsulfonyl)phenyl](nitroso)amino]benzoate;
4-[(4-nitrophenyl)(nitroso)amino]benzoic acid;
4-[(4-cyanophenyl)(nitroso)amino]benzoic acid;
4-[[4-(methylsulfonyl)phenyl](nitroso)amino]benzoic acid;
3-[(4-methoxyphenyl)(nitroso)amino]benzoic acid;
{4-[(4-methoxyphenyl)(nitroso)amino]phenyl}acetic acid;
1-{4-[(4-methoxyphenyl)(nitroso)amino]phenyl}ethanone;
4-[(4-fluorophenyl)(nitroso)amino]benzoic acid;
4-[(4-acetylphenyl)(nitroso)amino]benzoic acid;
4-[(3-fluorophenyl)(nitroso)amino]benzoic acid;
{4-[(3-fluorophenyl)(nitroso)amino]phenyl}acetic acid;
{4-[(4-fluorophenyl)(nitroso)amino]phenyl}acetic acid;
3-[(3-methoxyphenyl)(nitroso)amino]benzoic acid;
4-[(3-methylphenyl)(nitroso)amino]benzoic acid;
4-[(3-chlorophenyl)(nitroso)amino]benzoic acid;
3-[(4-acetylphenyl)(nitroso)amino]benzoic acid;
4-[[4-(methylsulfonyl)phenyl](nitroso)amino]benzonitrile;
3-[(4-acetylphenyl)(nitroso)amino]benzonitrile;
N-nitrosodiphenylamine-4,4′-dicarboxylic acid;
4-{nitroso[4-(trifluoromethoxy)phenyl]amino}benzoic acid;
4-[[4-(methylthio)phenyl](nitroso)amino]benzoic acid;
{4-[(4-chlorophenyl)(nitroso)amino]phenyl}acetic acid;
{4-[(3-methoxyphenyl)(nitroso)amino]phenyl}acetic acid;
4-[[4-(acetylamino)phenyl](nitroso)amino]benzoic acid;
4-[(3-cyanophenyl)(nitroso)amino]benzoic acid;
4-[[3-(aminocarbonyl)phenyl](nitroso)amino]benzoic acid;
(4-{nitroso[4-(trifluoromethyl)phenyl]amino}phenyl)acetic acid;
{4-[[4-(methylsulfonyl)phenyl](nitroso)amino]phenyl}acetic acid;
4-[(3-chlorophenyl)(nitroso)amino]benzonitrile;
4-{nitroso[4-(trifluoromethoxy)phenyl]amino}benzonitrile;
4-[(4-methoxyphenyl)(nitroso)amino]benzamide;
4-[(4-nitrophenyl)(nitroso)amino]benzamide;
4-[(4-fluorophenyl)(nitroso)amino]benzamide;
4-{nitroso[4-(trifluoromethoxy)phenyl]amino}benzamide;
1-{4-[(4-chlorophenyl)(nitroso)amino]phenyl}ethanone;
{4-[(4-fluorophenyl)(nitroso)amino]phenyl}methanol;
(4-{nitroso[4-(trifluoromethoxy)phenyl]amino}phenyl)methanol;
N-{4-[[4-(hydroxymethyl)phenyl](nitroso)amino]phenyl}acetamide;
{4-[(3-chlorophenyl)(nitroso)amino]phenyl}methanol;
{4-[(3-methylphenyl)(nitroso)amino]phenyl}methanol;
3-[(4-fluorophenyl)(nitroso)amino]benzoic acid.

24. Use according to claim 22, for the preparation of a medicament that is useful for the treatment of and preventing diabetes and/or metabolic insulin-resistance syndrome.

25. Use according to claim 22, for the preparation of a hypotriglyceridaemiant medicament.
